(19) United States
(12) Patent Application Publication
(10) Pub. No.: US 2023/0061363 A1
(43) Pub. Date: Mar. 2, 2023

(54) EMBOLIC DEVICE WITH IMPROVED NECK COVERAGE

(71) Applicant: INCUMEDx Inc., Newark, CA (US)

(72) Inventors: Berchell John Yee, Fremont, CA (US); Amiel Richard Aguilar, San Jose, CA (US)

(21) Appl. No.: 17/899,982

(22) Filed: Aug. 31, 2022

Related U.S. Application Data
(60) Provisional application No. 63/239,259, filed on Aug. 31, 2021.

(51) Int. Cl. A61B 17/12

(52) U.S. Cl. CPC A61B 17/12145; A61B 17/12113; A61B 2090/3966

(57) ABSTRACT

In various aspects, the invention includes an embolic device for use in treating a vascular disorder that can include a flexible structure including a series of alternating narrow portions and link portions. Each link portion can include two struts that substantially circumscribe an opening in at least one plane. In some cases, the embolic device further includes a link portion coil disposed over each of the two struts of at least one link portion.

### CROSS-REFERENCE TO RELATED APPLICATIONS

This application claims priority to U.S. Provisional Application No. 63/239,259, filed Aug. 31, 2021. The foregoing application is incorporated by reference herein in its entirety and for all purposes.

### TECHNICAL FIELD

In general, various embodiments of this invention relate to embolic devices for use in the minimally-invasive treatment of aneurysms and other vascular disorders and, more specifically, to an embolic device that can be configured (e.g. shaped) to accomplish improved filling and/or coverage of a neck of a vascular disorder.

### BACKGROUND

In general, an aneurysm is a swelling or bulge that forms a cavity in the wall of a blood vessel. One type of aneurysm is a cerebral aneurysm, which forms in an artery of the brain. A cerebral aneurysm may develop suddenly without initial symptoms, and can cause extreme pain. In general, in 15% of cerebral aneurysm cases, the patient dies suddenly upon development of the cerebral aneurysm; in another 15% of cerebral aneurysm cases, the patient dies under medical treatment; and in 30% of cerebral aneurysm cases, the patient survives after treatment but feels an acute aftereffect. As such, a cerebral aneurysm (or any aneurysm) is a very concerning development.

The treatment of aneurysms and other similar vascular disorders often involves the placement of microcoils within the cavity formed by the aneurysm or disorder. Doing so can cause blood to clot, prevent an additional inflow of blood, and decrease the risk of the aneurysm or disorder rupturing (i.e., an embolization). In order to be effective, an embolic microcoil must apply pressure sufficient to prevent additional inflow of blood, but not an excessive amount of pressure that causes rupture.

An important feature of an embolic device is its ability to block the aneurysm's neck, i.e., the opening where the aneurysm meets the blood vessel. Such blockage can be critical for ensuring that excessive amounts of blood do not flow into the aneurysm, risking further bulging or rupture. Prior approaches for blocking the aneurysm neck include covering the neck with stent-like or braided structures. While these approaches can sometimes be effective, there are still opportunities for improvement.

Accordingly, there is a need for an improved embolic device that achieves improved filling and/or blockage of a neck of an aneurysm.

### SUMMARY OF THE INVENTION

In various embodiments, the present invention relates to improved embolic devices and systems that achieve improved filling and neck blockage over conventional devices. In particular, the device features improved frame geometry over conventional device, which allows a greater exhibition of familiar bare platinum coil (BPC)-like behavior. In some instances, the device uses coiled structures that can be more compliant than braided mesh devices. Moreover, the increased porosity and simplification of the frame geometry can result in the device being compatible with smaller microcatheters (e.g., inner diameter (ID) in a range from 0.0135 inches to 0.0190 inches). In addition, in some embodiments, the coiled structures can extend along an entire or substantially an entire length of the device, which can enable improved visibility to e.g., surgeons or operators.

In general, in one aspect, embodiments of the invention feature an embolic device for use in treating a vascular disorder. The embolic device can include a flexible structure that includes a series of alternating narrow portions and link portions, each link portion including two struts that substantially circumscribe an opening in at least one plane. The embolic device can further include a link portion coil disposed over each of the two struts of at least one link portion.

In various embodiments, the structure can include two spaced apart elements that extend along substantially an entire length of the device. The two spaced apart elements form the two structs of each link portion, and/or are spaced more closely together in the narrow portions than the link portions. The structure can include a material including platinum, tantalum, nitinol, alloys thereof, and/or combinations thereof. In some cases, the structure includes a thickness in a range from 0.0005 inches to 0.027 inches. In some cases, each link portion includes a flat sheet, a thin film, and/or a drawn-filled tubing wire. In some cases, each link portion includes at least one of a diamond-like shape, circular, oval, rectangular, triangular, or polygonal shape.

In various embodiments, each link portion is adapted to compress when the embolic device is disposed within a microcatheter. Each link portion can be further adapted to expand upon deployment of the embolic device from the microcatheter. In some cases, the narrow portions and the link portions alternate with consistent spacing. In other cases, the narrow portions and the link portion alternate with inconsistent spacing. In some cases, the opening includes an open cell. In other cases, the opening includes a closed cell.

The embolic device can include a narrow portion coil disposed over at least one narrow portion. In some cases, the link portion coil and the narrow portion coil are continuous with each other. The continuous link portion coil and narrow portion coil extend along substantially an entire length of the device. In other cases, the link portion coil and the narrow portion coil are separate from each other. At least a portion of the embolic device is radiopaque.

In general, in another aspect, embodiments of the invention feature a method for treating a vascular disorder. The method can include the step of positioning within the vascular disorder an embolic device. The embolic device may include a flexible structure that includes a series of alternating narrow portions and link portions. Each link portion includes two struts that substantially circumscribe an opening in at least one plane. The embolic device may further include a link portion coil disposed over each of the two struts of at least one link portion. In various embodiments, method of treatments related to this aspect are also contemplated herein.

These and other objects, along with advantages and features of the embodiments of the present invention herein disclosed, will become more apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is schematic perspective view of an embolic device having a spiral shape disposed within an aneurysm, according to one embodiment of the invention;

FIG. 2A is a schematic top view of an embolic device having narrow portions and link portions, according to one embodiment of the invention;

FIG. 2B is a schematic side view of an embolic device having link portions including an open cell, according to one embodiment of the invention;

FIG. 2C is a schematic side view of an embolic device having link portions including a closed cell, according to one embodiment of the invention;

FIG. 3 is a picture of an embolic device having narrow portions, formed from coil segments, and link portions, according to one embodiment of the invention;

FIG. 4 depicts schematic side views of embolic devices having open cells closed by narrow portion coils, according to some embodiments of the invention;

FIG. 5 depicts schematic side views of embolic devices having one or more drawn filled tubing wires, according to some embodiments of the invention;

FIG. 6 depicts schematic side views of embolic devices having one or more drawn filled tubing wires, according to some embodiments of the invention; and

### DETAILED DESCRIPTION

Embodiments of the present invention are directed toward an improved design for an embolic device and methods of using the improved device. Neck blockage is an important function for embolic devices because it determines how much fluid can pass through the embolic device into the aneurysm, which can directly impact how effective the embolic device is in treating the vascular disorder. Embodiments of the present invention include embolic devices having shapes and/or configurations that accomplish improved neck blockage and other performance parameters over conventional devices.

In general, all of the embolic devices described herein can take any known form, e.g., a microcoil (e.g., bare platinum coil), flat sheet, thin film, drawn-filled tube or drawn filled tubing wire (DFT), combinations thereof, etc., even though in some instances a particular device may only be described herein as having one of these forms. In addition, all of the embolic devices described herein can be formed from any suitable material, e.g., shape memory material (e.g., nickel titanium, also known as “nitinol” as used herein), platinum, tantalum, alloys thereof, combinations thereof, etc., even though in some instances a particular device may only be described herein as being formed of one of these materials. Furthermore, in various instances, all of the embolic devices described herein can include a structure (e.g., microcoil, flat sheet, thin film, etc.) covered by a cover element, as described for example in U.S. Patent Publication No. US-2016-0022275-A1, which is incorporated herein by reference in its entirety. In some embodiments, the embolic devices described herein can have the function or take the form of those described for example in U.S. Patent Publication No. 2019-0307546-A1, which is incorporated herein by reference in its entirety.

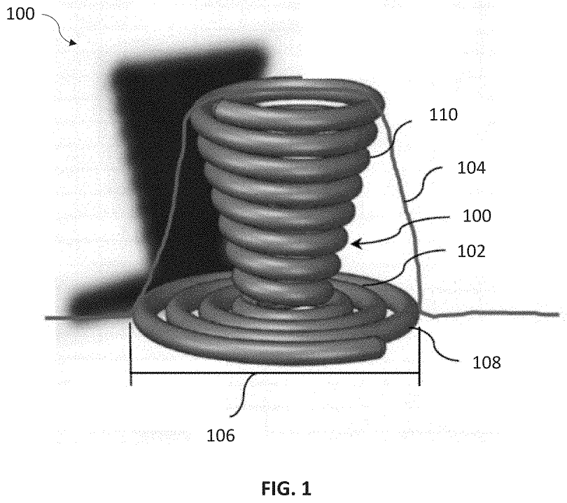

FIG. 1 is schematic perspective view of an embolic device 100 having a spiral shape disposed within an aneurysm, according to one embodiment of the invention. As shown in FIG. 1, the embolic device 100 can be used to treat a vascular disorder 104 that has a neck 106, e.g., an opening between a blood vessel and a cavity of the aneurysm 104. In some instances, the embolic device 100 can include a portion 108 disposed within and/or blocking the aneurysm neck 106 and another portion 110 disposed within the cavity of the aneurysm 104. In general, the portion 108 blocking the neck 106 can take any shape, e.g., a spiral shape as shown in FIG. 1. The spiral shape of the portion 108 can be formed in any suitable three dimensional shape, e.g., a disc (as shown in FIG. 1), a sphere, a semi-sphere (or partial-sphere), a cone, etc. The portion 108 having a spiral shape has been observed to accomplish improved blockage of the neck 106 over conventional devices. The portion 110 disposed within the cavity of the aneurysm 104 can also take any shape, which can be the same or a different shape as the portion 108 blocking the aneurysm neck 106. For example, as shown in FIG. 1, the portion 110 can have a spiral shape, but in other embodiments it can have other shapes, including random or non-geometric shapes. The spiral of the portion 110 can also be formed in any suitable three-dimensional shape, e.g., a disc, a sphere, a semi-sphere (or partial sphere), a cone (as shown in FIG. 1), etc.

One problem experienced with conventional devices is that their effectiveness in blocking an aneurysm neck is significantly affected by the orientation of the device upon delivery to a treatment site, which can sometimes be difficult to accomplish in a repeatable manner. Embodiments of the present invention solve this problem by featuring an embolic device that effectively blocks the aneurysm neck 106 regardless of its orientation upon placement into the aneurysm 104 or, in some cases, that blocks the aneurysm neck 106 in many more orientations than a conventional device (e.g., the majority of the orientations).

In various embodiments, embolic devices of the present invention can be formed from a flat sheet (e.g., formed from nitinol). In general, the flat sheet can be formed into any suitable shape. In some instances, the width of the flat sheet has a constant width. In other instances, the width of the flat sheet has a drafted or tapered (e.g., decreasing or increasing) width.

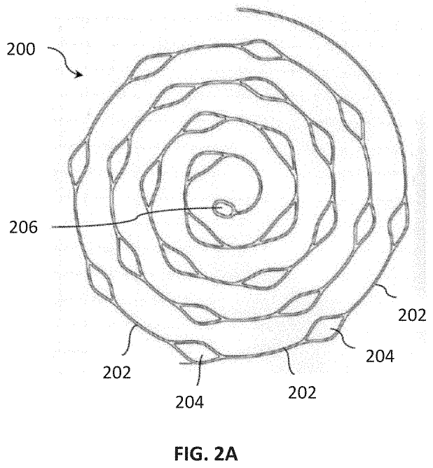

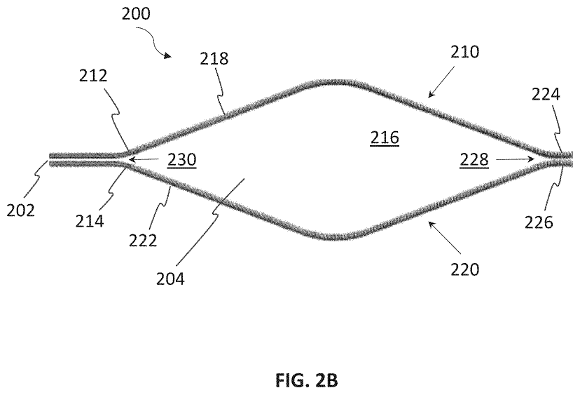

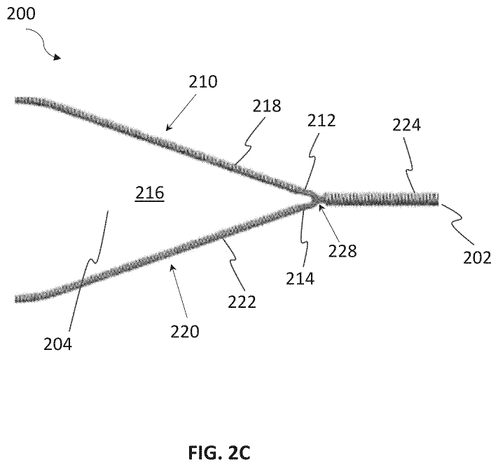

FIG. 2A is a schematic top view of an embolic device 200 having narrow portions and link portions, according to one embodiment of the invention; FIG. 2B is a schematic side view of an embolic device 200 having link portions including an open cell, according to one embodiment of the invention; FIG. 2C is a schematic side view of an embolic device 200 having link portions including a closed cell, according to one embodiment of the invention;

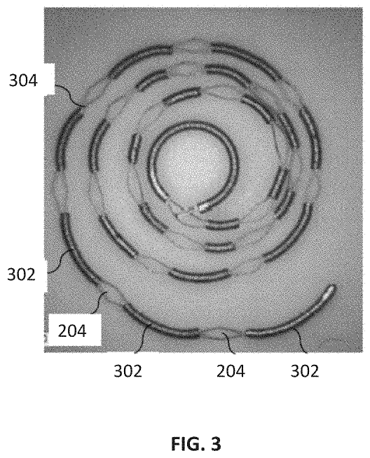

In various embodiments, an embolic device 200 can include a flexible structure that includes a series of alternating narrow portions 202 and link portions 204, as shown for example in FIG. 2A. In some embodiments, the narrow portions 202 and the link portions 204 alternate with consistent spacing. In other embodiments, the narrow portions 202 and the link portions 204 alternate with inconsistent spacing. The link portions 204 circumscribe an opening in at least one plane, e.g., the plane of the page, as shown in FIG. 2A. In general, the link portions 204 can have any suitable regular or irregular shape, e.g., diamond-like (e.g., as shown in FIGS. 2A and 3), circular, oval, rectangular, triangular, or polygonal shape (e.g., hexagon, pentagon, or quadrilateral) etc. In general, the embolic device 200 can be formed from any suitable structure, e.g., a coil, a flat sheet, a thin film, drawn filled tubing wire, single-material wires and combinations thereof, etc. For example, as shown in FIG. 2A, the narrow portions 202 can be formed of a single strip of flat sheet material (or multiple strips of flat sheet material with no opening in between) and the link portions 204 can be formed by at least two strips of flat sheet material that define a perimeter around or circumscribes an opening. The embolic device 200 (or a portion thereof) can also have any desirable thickness, e.g., in a range from 0.0001″ to 0.030″, in a range from 0.0005″ to 0.027″, in a range from 0.001″ to 0.025″, in a range from 0.002″ to 0.020″, in a range from 0.003″ to 0.015″, in a range from 0.004″ to 0.010″, in a range from 0.006″ to 0.008″. In another embodiment, the embolic device (or a portion thereof) has a thickness in a range from 0.002″ to 0.004″. In general, the flat sheet can be formed into any suitable shape. In some instances, the width of the flat sheet has a constant thickness. In other instances, the width of the flat sheet has a drafted or tapered (e.g., decreasing or increasing) thickness.

In various embodiments, as shown in FIG. 2B or 2C, the embolic device 200 includes two spaced apart elements 210, 220 that can extend along substantially an entire length of the embolic device 200. In some embodiments, the two spaced apart elements 210, 220 are spaced more closely together in the narrow portions 202 than the link portions 204. In such embodiments, each of the link portions 204 can include two struts 212, 214, formed by the two spaced apart elements 210, 220. The two struts 212, 214 of each link portion 204 may substantially circumscribe an opening 216 in at least one plane.

In some embodiments, the opening 216 is formed from an open cell, as shown for example in FIG. 2B. In other embodiments, the opening 216 is formed from a closed cell, as shown in FIG. 2C. As used herein, an “open cell” refers to a configuration in which the joints (e.g., joint 228 or 230 in FIG. 2B) on either or both ends of the link portion (e.g., link portion 204 in FIG. 2B) are not constrained. As used herein, a “closed cell” refers to a configuration in which the joints (e.g., joint 228 in FIG. 2C) on both ends of the link portion (e.g., link portion 204 in FIG. 2C) are constrained.

In various embodiments, the embolic device 200 can further include a link portion coil (e.g., link portion coil 218 or 222) and/or a narrow portion coil (e.g., narrow portion coil 224 or 226). The link portion coil can be disposed over each strut (e.g., strut 212 or 214) of at least one link portion 204 (or a portion thereof). The narrow portion coil can be disposed over at least one narrow portion 202 (or a portion thereof). In other embodiments, the link portion coil and the narrow portion coil are continuous with each other, as shown for example in FIG. 2B. The continuous link portion coil and narrow portion coil can extend along substantially an entire length of the embolic device, or in some cases along certain portions thereof. In some embodiments, the link portion coil and the narrow portion coil are separate from each other, as shown for example in FIG. 2C. In particular embodiments, the link portion coil and/or the narrow portion coil includes nitinol, platinum, tantalum, alloys thereof, or combinations thereof. For example, the link portion coil can be a platinum coil, and the narrow portion coil can be a nitinol coil. In another example, the link portion coil and the narrow portion coil are drawn filled tubing wires. In some embodiments, the embolic device does not include any link portion coil and/or the narrow portion coil.

In various embodiments, at least a portion of the embolic device as described herein is radiopaque. In use, the embolic device can be positioned within a vascular disorder to achieve a desired outcome where implant performance and manufacturability are enhanced to facilitate intrasaccular wide neck support for embolic coils. The use of an open cell configuration, e.g., as shown in FIG. 2B, can allow for ease of loading coils onto both struts of the frame structure. In some embodiments, upon loading, the embolic device can be welded at a proximal end with a proximal tab that connects with a delivery pusher system.

In some instances, as shown for example in FIG. 3, the narrow portion coil (e.g., narrow portion coils 224 or 226 in FIG. 2B or FIG. 2C) may be coil segments 302. In some cases, the coil segments 302 are disposed over another structure (e.g., a flat sheet, thin film, etc.). In other cases, the coil segments 302 are not disposed over another structure. In general, the coil segments 302 can be attached to the link portions 204 using any known technique, e.g., melting a suture on the ends of each coil segment 302. Melting a suture on the ends of each coil segment 302 can also keep the coil segment 302 positioned between the link portions 204. In some instances, the link portions 204 can be fixedly attached to proximate narrow portions and a strain relief element 304 can be used to relieve strain between the portions. The strain relief element can be formed of any suitable material, e.g., a suture material, a melted polymer (e.g., polypropylene, polyethylene, high density polyethylene, low density polyethylene, polyurethane, polyether block amide, polyamides, polymer adhesives, etc.), etc.

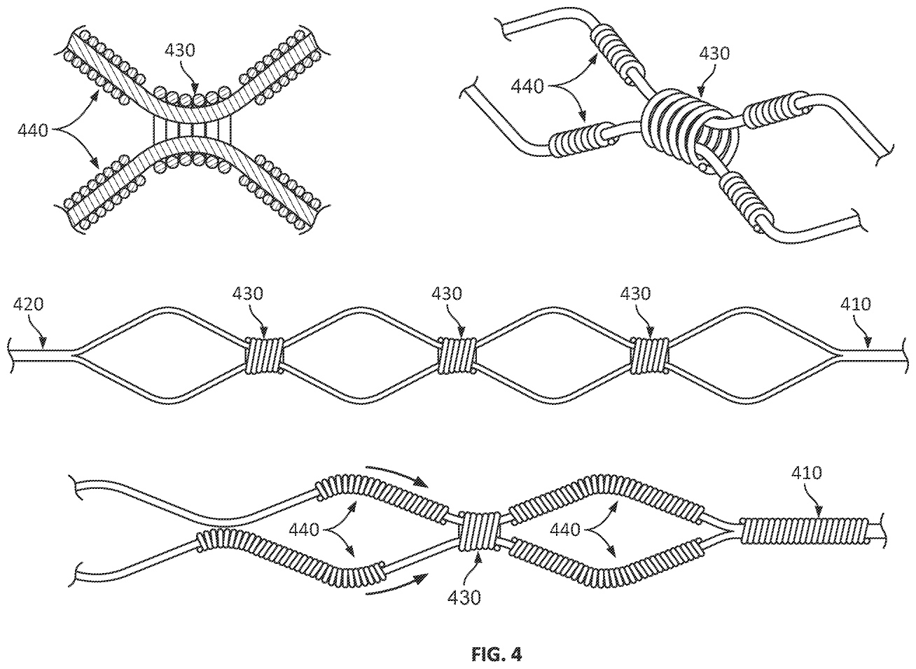

FIG. 4 depicts schematic side views of embolic devices having open cells closed by narrow portion coils, according to some embodiments of the invention. In some embodiments, as described further below, an open cell (or a portion thereof) can be substantially closed by holding (e.g., cuffing, collaring, welding, sealing, or attaching, etc) the joints (e.g., joints 228, 230 in FIG. 2B) of the open cell, and/or the entire narrow portion (or a portion thereof), while retaining a structure that is used to provide strut coverage across the aneurysm neck. In general, selection of open cells, closed cells, or a hybrid device including both open and closed cells can be based upon performance characteristics of each cell type. In general, in some embodiments, open cells can result in a more flexible and movable shape or device, while closed cells can result in a more rigid shape or device.

In particular embodiments, the embolic device is configured to include a closed cell at a distal end 410 of the embolic device, while the device include open cells in the remaining portion of the device including a proximal end 420. As shown in FIG. 4, an open cell of the device can be converted to a closed cell by providing a narrow portion coil (e.g., nitinol coils 430) to attach the narrow portion that includes the joint of the open cell, resulting in a cell structure that is close to the monolithic or single strut configuration of the narrow portion e.g., as shown in FIG. 2C. Further, a link portion coil (e.g., platinum coils 440 loaded from the proximal end 420 of the embolic device) can be provided and extend to certain portions or segments along the struts of the embolic device.

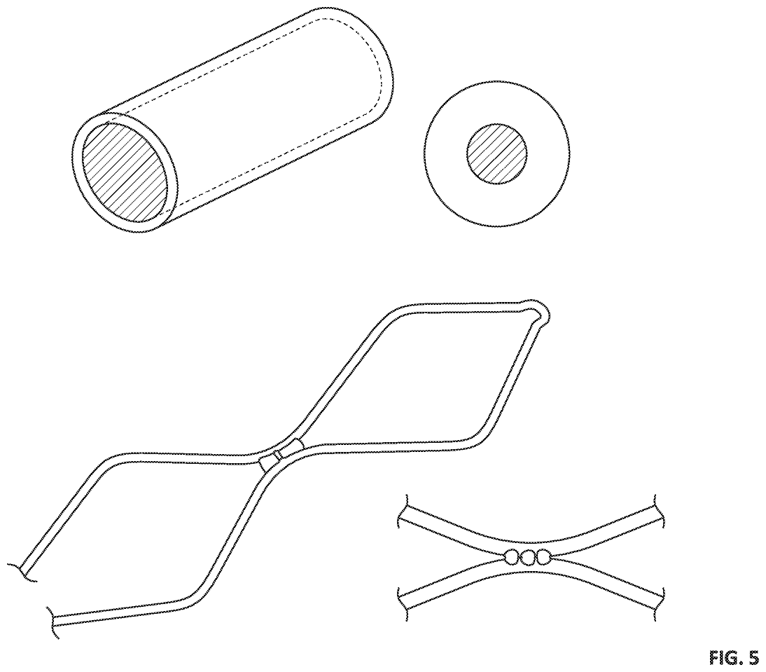

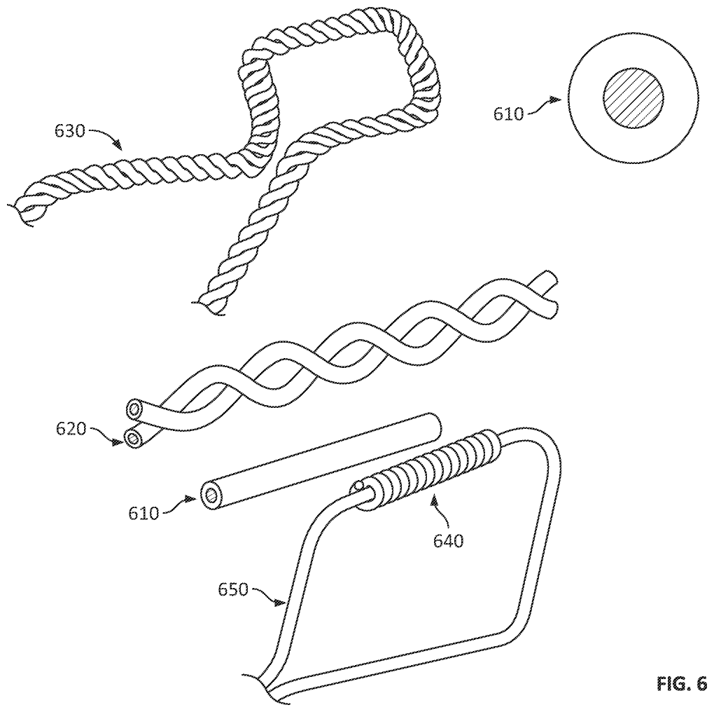

FIGS. 5 and 6 depict schematic side views of embolic devices having one or more drawn filled tubing wires, according to some embodiments of the invention.

In various embodiments, the embolic device, as shown in FIG. 5 or 6, can include one or more drawn-filled tubes or drawn filled tubing wires (DFTs). As used herein, “drawn-filled tube,” “drawn filled tubing wire,” “DFT,” “DFT wire,” “DFT coil,” or similar terms refers to a structure (e.g., a wire, or a tubing) that includes an outer shell filled with a core, where the outer shell and the core include different materials or compositions. In particular embodiments, the drawn filled tubing wire as described herein includes platinum in the core and nitinol in the outer shell, or a reversed structure (e.g., platinum as the material in the outer material and nitinol as the core material). Advantageously, by using such drawn filled tubing wire(s) which already includes platinum either in the core or in the outside shell, no additional platinum coils (e.g., platinum coils 440) are needed. Accordingly, a relative large outer diameter of the link portion or the strut can be achieved. In various other embodiments, any description herein of use of a DFT wire can be replaced with a single material/composition wire.

In various embodiments, the drawn filled tubing wire can include any desired composition, e.g., about 1%, 5%, 10%, 20%, 30%, 40%, 50%, 60% 70%, 80%, 90%, or 99% platinum, tantalum, or their combinations thereof by weight. In particular embodiments, the drawn filled tubing wire includes about 10%, 20%, 30% platinum or tantalum by weight.

In various embodiments, the drawn filled tubing wire can include any desirable outside diameter (or outer diameter), e.g., in a range from 0.0001″ to 0.1″, 0.0002″ to 0.09″, 0.0003″ to 0.08″, 0.0004″ to 0.07″, 0.0005″ to 0.06″, 0.0006″ to 0.05″, 0.0007″ to 0.04″, 0.0008″ to 0.03″, 0.0009″ to 0.02″, 0.001″ to 0.01″, 0.002″ to 0.009″, 0.003″ to 0.008″, 0.004″ to 0.007″, 0.005″ to 0.006″. In particular embodiments, the drawn filled tubing wire includes an outside diameter (or outer diameter) of about 0.0025″, 0.0035″, 0.0045″, 0.0055″, or 0.0065″.

In various embodiments, the drawn filled tubing wire can include any desirable interior diameter (or inside diameter), e.g., in a range from 0.0001″ to 0.1″, 0.0002″ to 0.09″, 0.0003″ to 0.08″, 0.0004″ to 0.07″, 0.0005″ to 0.06″, 0.0006″ to 0.05″, 0.0007″ to 0.04″, 0.0008″ to 0.03″, 0.0009″ to 0.02″, 0.001″ to 0.01″, 0.002″ to 0.009″, 0.003″ to 0.008″, 0.004″ to 0.007″, 0.005″ to 0.006″. In particular embodiments, the drawn filled tubing wire includes an interior diameter (or inside diameter) of about 0.00075″, 0.00105″, 0.00135″, 0.00165″, or 0.00195″.

In various embodiments, the outside diameter and/or the interior diameter of the drawn filled tubing is evaluated by measuring the radiopacity of the embolic device, and may be adjusted e.g., to provide sufficient visibility in radiographic images to e.g., surgeons or operators.

As shown in FIG. 5, in some embodiments, each spaced apart element of the embolic device includes a DFT, and the DFT may be welded at the narrow portion to provide a substantially closed cell. In various embodiments, the weld in the foregoing sentence can be replaced with any known attachment scheme. For example, in some embodiments, a separate coil can be wrapped about the spaced apart elements 210, 220 at the narrow portion. In some cases, the spaced apart elements 210, 220 may not be physically adhered at the narrow portion at all, but rather disposed in contact or proximate each other. In such cases, and in general, any spacing can be used, e.g., any spacing narrower than the widest spacing between the spaced apart elements in the link portion 204, e.g., up to 0.1%, up to 0.5%, up to 1%, up to 5%, up to 10%, up to 30%, up to 50%, up to 75%, and up to 90% of the widest spacing between the spaced apart elements in the link portion 204.

As shown in FIG. 6, in some embodiments, the spaced apart element of the embolic device includes a single DFT 610 (e.g., a core including platinum and an outside shell including nitinol). In some embodiment, the DFT is configured to increase the amount of material (e.g., platinum) at each spaced apart element such that a greater radiopacity can be achieved. For example, the spaced apart element 620 of the embolic device 630 can include two or more DFTs that are e.g., twisted. In another example, a DFT 640 (e.g., in a coil form) can be disposed at another DFT 650 (e.g., forming the spaced apart element).

In various embodiments, the embolic devices described herein can be introduced, delivered, positioned, and implanted within a vascular disorder using a microcatheter. The microcatheter can be a flexible, small diameter catheter having, for example, an inside diameter between 0.015 inches and 0.035 inches (e.g., between 0.016 inches and 0.021 inches). The microcatheter may be introduced by an introducer sheath/guiding catheter combination placed in the femoral artery or groin area of a patient. In some instances, the microcatheter is guided into the vascular disorder with guidewires (e.g., long, torqueable proximal wire sections with more flexible distal wire sections designed to be advanced within tortuous vessels). Such guidewires may be visible using fluoroscopy and may be used to first access the vascular disorder, thereby allowing the microcatheter to be advanced over it into the disorder.

In some instances, once the tip of the microcatheter has accessed the vascular disorder, the guidewire is removed from the catheter lumen. The embolic device may then be placed into the proximal open end of the microcatheter and advanced through the microcatheter with a delivery mechanism. The embolic device may attach to a delivery mechanism via any suitable structure (e.g., a loop 206 in FIG. 2A) on a proximal end of the device. In some instances, while the embolic device is disposed within the lumen of the microcatheter it is in a straightened out form. A user (e.g., a physician) may advance and/or retract the embolic device several times to obtain a desirable position of the embolic device within the disorder. Once the embolic device is satisfactorily positioned, it can be released into the disorder. Upon release, the device may form its deployed shape, for example the spiral shapes described above, or any other desired configuration. In some instances, the formation of the shape upon deployment into the vascular disorder is caused by the shape-memory nature of the material used to form the embolic device (e.g., nitinol, platinum, tantalum, combinations thereof, or alloys thereof).

In use, the structure of alternating narrow portions 202 and link portions 204 can be positioned into a substantially spherical pattern, e.g., upon the deployment of the embolic device from the microcatheter, as shown in FIG. 2A, as shown in FIG. 2A, to prevent coils from herniating into the parent vessel of the anatomy. The shaped structure acts as a cage that provides coil retention in spherical octants, while featuring microcatheter re-accessibility. With this complete coverage, the embolic device 200 can also be used in ruptured aneurysms to provide support in a dome region.

Further explanation regarding the shape of the embolic devices described herein at various stages of the delivery process is instructive. The embolic device are generally manufactured to have a particular shape in an unconstrained configuration, e.g., as the device would exist in packaging or an operating room before being delivered to a patient. The particular shape can include any of the embolic device shapes described herein. During delivery, the embolic device is straightened out so that it can fit within and be delivered through a microcatheter (as described above). Once deployed out of the microcatheter to the vascular disorder, the embolic device can reform the shape it was manufactured to have (e.g., aided by a shape memory material). However, in some instances, the embolic device may not reform exactly into the shape it was manufactured to have, based on constraints imposed by the vascular disorder and other surrounding structures.

In various embodiments, the link portions (e.g., link portion 204 in FIG. 2) can compress or collapse (e.g., the openings can become narrower) when the embolic device (e.g., embolic device 200) is located in a microcatheter during delivery. The link portions can then expand (e.g., the openings can become wider) upon deployment of the embolic device to the vascular disorder. This can allow the embolic device to be more easily delivered with less friction through a microcatheter, while also effectively blocking the neck of the aneurysm upon deployment. In some instances, the narrow portion coil (e.g., coil segments 302 in FIG. 3) can further reduce friction of the embolic device during delivery through a microcatheter. As one example, the shape of the coil segments 302 can better match the shape of a lumen of the microcatheter. As another example, the coil segments 302 can increase the flexibility and malleability of the embolic device. As another example, the coil segments 302 can be formed of a material that generates less friction with the interior surface of the microcatheter. The coil segments 302 can also be formed of a radiopaque material (e.g., platinum) such that the embolic device can be viewed during delivery; for example, if the link portions are formed from a non-radiopaque material.

### Definitions

Unless expressly described elsewhere in this application (e.g., the use of the word “substantially” with respect to a geometric shape), as used herein, when the term “substantially” or “about” is before a quantitative value, the present disclosure also includes the specific quantitative value itself, as well as a ±10% variation from the nominal value unless otherwise indicated or inferred.

The term “about” refers to a ±10% variation from the nominal value unless otherwise indicated or inferred.

The term “each” include a portion of referents unless the context clearly dictates otherwise.

It must be noted that, as used in the specification, the singular forms “a,” “an” and “the” include plural referents unless the context clearly dictates otherwise.

The term “drawn-filled tube,” “drawn filled tubing wire,” “DFT,” “DFT wire,” “DFT coil,” or similar terms refers to a structure (e.g., a wire, or a tubing) that includes an outer shell filled with a core, where the outer shell and the core include different materials or compositions.

All references, issued patents and patent applications cited within the body of the specification are hereby incorporated by reference in their entirety, for all purposes.

Having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. An embolic device for use in treating a vascular disorder, the embolic device comprising:
a flexible structure comprising a series of alternating narrow portions and link portions, each link portion comprising two struts that substantially circumscribe an opening in at least one plane; and
a link portion coil disposed over each of the two struts of at least one link portion.

2. The embolic device of claim 1, wherein the structure comprises two spaced apart elements that extend along substantially an entire length of the device.

3. The embolic device of claim 2, wherein the two spaced apart elements form the two structs of each link portion.

4. The embolic device of claim 2, wherein the two spaced apart elements are spaced more closely together in the narrow portions than the link portions.

5. The embolic device of claim 1, wherein the structure comprises a material selected from the group consisting of platinum, tantalum, nitinol, alloys thereof, and combinations thereof.

6. The embolic device of claim 1, wherein the structure comprises a thickness in a range from 0.0005 inches to 0.027 inches.

7. The embolic device of claim 1, wherein each link portion comprises at least one of a flat sheet, a thin film, and a drawn-filled tubing wire.

8. The embolic device of claim 1, wherein each link portion comprises at least one of a diamond-like, circular, oval, rectangular, triangular, or polygonal shape.

9. The embolic device of claim 1, wherein each link portion is adapted to compress when the embolic device is disposed within a microcatheter.

10. The embolic device of claim 9, wherein each link portion is further adapted to expand upon the deployment of the embolic device from the microcatheter.

11. The embolic device of claim 10, wherein the series of alternating narrow portions and link portions are adapted to form a spherical shape upon the deployment of the embolic device from the microcatheter.

12. The embolic device of claim 1, wherein the narrow portions and the link portions alternate with inconsistent spacing.

13. The embolic device of claim 1, wherein the opening comprises an open cell.

14. The embolic device of claim 1, wherein the opening comprises a closed cell.

15. The embolic device of claim 1, further comprising a narrow portion coil disposed over at least one narrow portion.

16. The embolic device of claim 15, wherein the link portion coil and the narrow portion coil are continuous with each other.

17. The embolic coil of claim 16, wherein the continuous link portion coil and narrow portion coil extend along substantially an entire length of the device.

18. The embolic device of claim 15, wherein the link portion coil and the narrow portion coil are separate from each other.

19. The embolic device of claim 1, wherein at least a portion of the embolic device is radiopaque.

20. A method of treating a vascular disorder, the method comprising the step of:
positioning within the vascular disorder an embolic device comprising:
a flexible structure comprising a series of alternating narrow portions and link portions, each link portion comprising two struts that substantially circumscribe an opening in at least one plane; and
a link portion coil disposed over each of the two struts of at least one link portion.
